(19) United States
(12) Patent Application Publication
(10) Pub. No.: US 2009/0054536 A1
(43) Pub. Date: Feb. 26, 2009

(54) PALATABLE SUPPORT OF IMPROVED FORMULA

(75) Inventor: Jean-Pierre Maloisel, Toulouse (FR)

Correspondence Address: OLIFF & BERRIDGE, PLC, P.O. BOX 320850, ALEXANDRIA, VA, 22320-4850, US

(73) Assignee: VETINNOV, RAMONVILLE ST. AGNE (FR)

(21) Appl. No.: 12/278,230

(22) PCT Filed: Feb. 5, 2007
(86) PCT No.: PCT/FR2007/050750

§ 371 Date: Sep. 30, 2008

(30) Foreign Application Priority Data
Feb. 7, 2006 (FR) 06 01090

(51) Int. Cl. A61K 47/36; A61K 47/42

(52) U.S. Cl. 514/773; 514/778

(57) ABSTRACT

A palatable support is intended to at least partially wrap an active substance that is initially independent of the support. The support of the invention is remarkable in that it includes, as a percentage by weight based on the total weight of the support, 3% to 50% of glucose syrup, the dextrose equivalent (DE) of the glucose syrup being in the range 5 to 60.

The invention relates to a palatable support suitable for wrapping an active substance intended for an animal, in particular for dogs, cats, horses, pigs, and ruminants, and the invention also relates to methods of manufacturing and using said support.

Oral administration of active substances, in particular drugs, to animals is often difficult because some such substances taste bad and because the sense of smell and taste in such animals is highly developed.

In the event of non-ingestion, the chances of success of a veterinary treatment can drop dramatically.

Thus, there is a need to package active substances in a manner that can put animals, in particular dogs, cats, horses, pigs, and ruminants, off their guard.

European patent EP-A-1 562 549 discloses a palatable support for an active substance intended for an animal, in particular for use with a drug. The matrix of said support includes at least one sticky surface that is sufficiently adhesive to attach the active substance merely by contact between the active substance and the sticky surface. It suggests the use of a matrix that is “neutral”, i.e. substantially without taste, so that the same matrix can be used for different types of animal. However, there is no description of an embodiment of such a support.

Thus, there is a need for a palatable support in which an active substance can be embedded and that is suitable for different types of animal, in particular dogs and cats, and for a method of manufacturing said support.

The aim of the invention is to provide such a support.

In accordance with the invention, this aim is achieved by a palatable support intended to at least partially wrap an active substance that is initially independent of the support, remarkable in that it includes, as a percentage by weight based on the total weight of the support, 3% to 50% of glucose syrup, the dextrose equivalent (DE) of the glucose syrup being in the range 5 to 60, preferably in the range 5 to 40 and more preferably in the range 5 to 15, and, preferably, at least 2%, preferably at least 4%, more preferably at least 5% of rice starch, preferably hydrolyzed. Preferably, the percentage by weight of said rice starch is less than 20%, preferably less than 10%, more preferably less than 7%.

The term “initially independent active substance” means that on manufacture, the support does not contain the active substance. The term “active substance” means any substance that may provide treatment, in the medical and/or cosmetic and/or nutritional sense, prepared in the form of tablets, lozenges, gelules, pills, or powders, for example.

As can be seen in greater detail below in the description, the use of a glucose syrup with such a dextrose equivalent and in such a quantity means that a matrix can be manufactured that is acceptable both to cats and to dogs.

Further, the presence of rice starch renders the support sticky. Advantageously, the active substance adheres to it effectively. Combined with glucose syrup, the rice starch allows a homogeneous, pasty, and sticky support to be constituted that is effective in enabling active substances to be ingested, both by cats and dogs.

Unless otherwise indicated, all of the percentages are percentages by weight, based on the total weight of the support.

Preferably, the support exhibits one or more of the following optional characteristics:

The support includes a moistening agent selected from alcohols or a mixture thereof, preferably from propylene glycol, glycerol, sorbitol, or a mixture thereof. A quantity of a mixture of propylene glycol and glycerol of 3% or more and/or 15% or less is preferable, the quantity of propylene glycol preferably being 3% or more, preferably 5% or more, and/or 15% or less, preferably 10% or less, the quantity of glycerol preferably being 5% or more, more preferably 9% or more, and/or 20% or less, preferably 15% or less. A quantity of sorbitol of 3% or more, preferably 5% or more, and/or 15% or less, preferably 10% or less, is also preferable. Preferably again, the support comprises a quantity of 1% or more of lecithin, preferably 3% or more, and/or 15% or less, preferably 8% or less.

Advantageously, these characteristics can produce a water activity (Aw) of the support of less than 0.75 and a shelf life that can be as long as about 18 months.

The support includes a quantity of proteins of animal origin, preferably selected from the group formed by poultry, pork, and fish meal, and mixtures thereof, which is preferably 5% or more, more preferably 30% or more, and/or 90% or less, preferably 50% or less.

The support includes modified starch, preferably modified rice starch, preferably AXGFP (AX finest grade precuit [pre-cooked]) sold by the supplier REMY Industries (Belgium) that is pre-hydrolyzed rice starch, in a quantity that is preferably 4% or more, and/or 90% or less, preferably 50% or less, more preferably 20% or less.

The rice starch is cooked. Preferably, it contains more than 90% by weight, preferably more than 95% by weight, of amylopectin.

Preferably, the rice starch is waxy, i.e. it contains substantially only amylopectin. By comparison, a “regular” starch only contains about 80% amylopectin. Advantageously, the high amylopectin content makes the support very sticky and allows its glucose content to be limited.

The support includes a quantity of fatty substances, preferably selected from oils, fats, emulsifying agents, or a mixture thereof. A quantity of oil, preferably selected from vegetable oils, in particular rapeseed oils and/or sunflower seed oils, of 0.01% or more, preferably 1% or more, and/or less than 10%, preferably less than 5%, is desirable. A quantity of fat, preferably of animal origin, in particular duck and/or pork fat, of 0.01% or more and/or 10% or less, preferably 2% or less, is desirable. A quantity of emulsifying agent of 0.01% or more, and/or 10% or less, preferably 1% or less, is also desirable, the preferred emulsifying agent being 2901 sold by the supplier Brenntag Nev.

The support includes preservatives, preferably constituted by BHA (butylated hydroxyanisole) and/or propyl gallate and/or citric acid and/or lactic acid and/or propenoic acid and/or sorbic acid and/or phosphoric acid. A total quantity of preservatives of 0.01% or more and/or 10% or less, preferably 3% or less, is preferred. Preferred preservatives are selected from Thermox™, Pet SAVOR®, sorbate, or a mixture thereof. A quantity of Thermox™ of 0.01% or more and/or 2% or less, preferably 1% or less, is desirable. A quantity of Pet SAVOR® of 0.5% or more, preferably 0.7% or more, and/or 5% or less, preferably 2% or less, is also desirable. A quantity of sorbate, preferably potassium sorbate, of 0.01% or more, preferably 0.1% or more and/or 5% or less, preferably 1.5% or less, is also desirable.

The support includes a quantity of a palatability enhancer, preferably specific to the type of animal for which the support is intended and selected from Superpremium type products, preferably based on pig liver, more preferably 8P enhancers for dogs and 9P or 9M for cats sold by SPF Diana, of 1% or more, preferably 2% or more and/or 15% or less, preferably 10% or less, more preferably 6% or less.

The percentage by weight of glucose syrup is more than 5% and/or less than 20%, preferably less than 15%, more preferably less than 10%, or even less than 7%, or even less than 6.5%.

The dextrose equivalent (DE) of the glucose syrup is more than 10, preferably more than 15, even more. preferably more than 18 and/or less than 25, preferably less than 22.

The invention also provides a method of manufacturing a palatable support intended to facilitate ingestion of an active substance by an animal, said method comprising a step in which various constituents are mixed and being remarkable in that said constituents and their quantities are determined so as to obtain a support in accordance with the invention.

Finally, the invention provides a method of packaging an active substance within a support, said active substance initially being independent of said support and preferably in the form of a pill, lozenge, gelule, tablet or powder, in which the user at least partially wraps said active substance in said support, said method being remarkable in that the support is in accordance with the invention.

Other characteristics and advantages of the present invention become apparent from the following description and from the accompanying drawings in which:

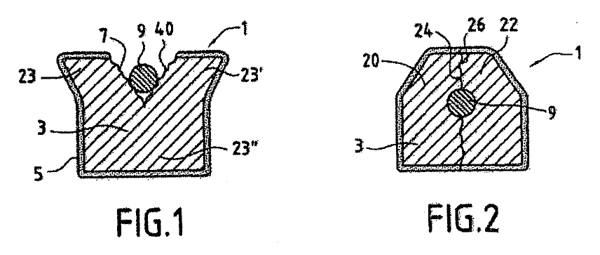

FIG. 1 represents a cross-sectional view of a support in accordance with the invention; and

FIG. 2 represents a cross-sectional view of the support shown in FIG. 1, after wrapping a tablet with the support.

In the various figures, identical reference numerals have been used to designate objects that are identical or analogous.

FIG. 1 shows a support 1 comprising a matrix 3 at least partially covered with a layer 5 formed from a palatable material or “palatability enhancer”. The support 1 is provided in the form of a substantially homogeneous cube which the user tears into two pieces 20 and 22.

In FIG. 1, the support 1 is shown in an intermediate condition between its substantially cubic initial condition and its condition when torn into two pieces 20 and 22. In said intermediate condition, the matrix 3 includes two extremities 23 and 23′ connected via a common base 23″. Once torn, the matrix 3 exposes a contact surface 27 formed by surfaces 29 and 28 of pieces 20 and 22 respectively, defining the tear.

Advantageously, the layer 5 of palatability enhancer and the bulk shape of the cube protects the matrix 3 from drying out. The service life of the support 1 is thus augmented.

The matrix 3 is preferably a substance that is plastically deformable, i.e. it substantially retains the shape which it is given.

The composition of matrix 3 is as described below.

A highly preferred composition for the support 1 is as follows, as a percentage by weight based on the total weight of the support:
in the range 41.5% to 42.5% of poultry meal, preferably about 41.9%;
in the range 4% to 5% of a palatability enhancer, preferably of the Superpremium type sold by SPF Diana, preferably about 4.5%;
in the range 5.3% to 6.3% of hydrolyzed rice starch, preferably of the AXFGP type, preferably about 5.8%;
in the range 0 to 1% of sodium chloride, preferably about 0.7%;
in the range 0.01% to 1% of sorbate, preferably about 0.7%;
in the range 0.01% to 0.1% of Thermox™, preferably about 0.08%;
in the range 11% to 12% of glycerol, preferably about 11.5%;
in the range 7% to 8% of propylene glycol or sorbitol, preferably about 7.7%;
in the range 5.5% to 6.5% of glucose syrup with a dextrose equivalent (DE) in the range 8 to 22, preferably about 5.8% of glucose syrup with a dextrose equivalent (DE) of about 20;
in the range 2% to 3% of oil, preferably sunflower and/or rapeseed oil, preferably about 2.6%;
in the range 0.01% to 1% of fats, preferably duck and/or pork, preferably about 0.6%;
in the range 0.6% to 1.6% of Pet SAVOR®, preferably about 1.2%;
in the range 5.2% to 6.2% of lecithin, preferably soya, preferably about 5.7%;
in the range 10.3% to 11.3% of water, preferably about 10.8%; and
in the range 0.01% to 1% of emulsifying agent, preferably about 0.4%.

Surprisingly, said matrix is well accepted both by dogs and by cats. It is plastic and has a sticky nature that allows the pieces 20 and 22 to be aggregated by simple manual pressure. The sticky nature allows attachment of the tablet 9.

The palatability enhancer, preferably Superpremium™, may be present on the surface of the matrix 3 in the form of a layer, or it may be contained directly within the matrix 3.

A preferred method of manufacturing a support in accordance with the invention comprises the following various steps:

a) mixing the constituents in the form of powders, in particular glucose syrup when in the powder form (if it is in the liquid form it will be incorporated in step b)), and/or proteins of animal origin and/or starch and/or sorbate and/or Thermox™ and/or salt, to obtain a homogeneous powder;

b) mixing the liquid and/or pasty compounds, in particular a moistening agent and/or the fats and/or a preservative and/or the lecithin and/or the water, with said homogeneous powder;

c) leaving in the cold under conditions that are well known to the skilled person to facilitate passage through a machine, followed by extrusion, still in the cold, in particular using a single screw “paste machine” type extruder or pusher, to obtain a matrix;

d) optionally, depositing the palatability enhancer on said matrix, to form a continuous or, preferably, discontinuous layer. In the such circumstances, there exist zones in the matrix that are not covered by the enhancer to allow the support to be handled without the fingers coming into contact with the flavor enhancer. The flavor enhancer may give off an odor that is disagreeable to human beings.

The support obtained may then be packed, preferably in the form of individually packed cubes, to limit their drying off and facilitate their handling. The support covered with the palatability enhancer may be pre-slit, pierced or grooved to pre-define future contact surfaces with the active substance. The palatability enhancer used in step d) is preferably specific to the animal for which the support is intended.

However, in accordance with the invention, in steps a) and b), a proportion of in the range 3% to 50% of glucose syrup with a DE in the range 5 to 60 is selected in order to obtain, at the end of step c), a matrix that is suitable for both cats and dogs. The production costs are advantageously reduced thereby. The palatability enhancer may be introduced during step a) and/or b); since all of the other constituents are acceptable to cats and dogs, the manufacturing costs are advantageously kept reduced. It should also be noted that the method of manufacturing the support of the invention means that all cooking steps during manufacturing can be dispensed with. Preferably, the manufacturing method of the invention does not include a cooking step between steps a) and d) inclusive.

The various steps and the order in which they have been described are provided by way of non-limiting illustrative example. In particular, it is possible for steps a) and b) to be reversed. Similarly, it is possible for the palatability enhancer to be introduced into the matrix during one of steps a) or b).

In order to package a tablet 9 using a support of the invention such as that represented in FIG. 1, a user may proceed as follows.

The user tears the support 1 into two pieces 20 and 22. On opening the matrix, the user exposes surfaces 28 and 29.

The user then positions the tablet 9 on one of surfaces 28 and 29 and disposes the pieces 20 and 22 of the torn support 1 so that the surfaces 28 and 29 are substantially facing each other. The user then exerts an action to press one of these pieces against the other in order to aggregate them. It is also possible to deform the matrix manually to increase the contact surface area between the tablet 9 and the contact surfaces 28 and 29.

Preferably, the contact surface covers substantially all of the outer surface of the tablet 9 in order to enclose it.

The surfaces 28 and 29 of the support of the invention are such that after they have been pressed against each other, they no longer spontaneously separate from each other. The term “spontaneously” means “under the sole effect of the elasticity of the matrix”, i.e., without the user's intervention.

Preferably, the tear is sufficiently wide to allow the tablet 9 to be inserted completely. Advantageously, the support 1 of the invention can thereby completely encapsulate, wrap or enclose the tablet 9 after reconstituting the initial cube. No repellant odor or disagreeable taste can be released from the tablet 9 inserted in the support 1.

Clearly, packaging an active substance using a support in accordance with the invention is very simple and may be carried out extemporaneously on single units by a user. No equipment is required.

Advantageously, the same procedure for packaging and the same supports 1 may be used for active substances of very different forms.

Advantageously, the user can package several active substances that may be in different forms (gelule, tablet, lozenge, powder, etc) using the same support 1.

The effectiveness of a support in accordance with the invention in putting animals off their guard has been tested on dogs and cats. The test supports had the following composition:
in the range 41% to 42% of poultry meal;
in the range 4.5% of Superpremium type palatability enhancer;
about 5.8% of AXFGP;
about 0.64% of sodium chloride;
about 0.64% of sorbate;
about 0.07% of Thermox™;
about 11.5% of glycerol;
about 7.67% of propylene glycol;
in the range 5.7% to 5.85% of glucose syrup with a dextrose equivalent (DE) of 20;
about 2.57% of a mixture of oil, fats and 2901 emulsifying agent from Brenntag;
about 1.24% of Pet SAVOR®;
about 5.76% of soya lecithin;
about 10.8% of water.

The palatability enhancer is not just included in the mixture of powders but is also deposited on the outside of the matrix 3 to form a layer 5 around the support 1. The support is thus non sticky, even after tearing into several pieces. Further, the periphery of the support is particularly palatable, which is of particularly advantage when the animal is going to sniff the support.

The tests were carried out under the same conditions for the dogs and for the cats. Two times a day over 5 days, a tablet wrapped in the support was presented to the animal. Each time, the animal could either eat neither the support nor the tablet, or eat the support but spit out the tablet, or eat the support and the tablet.

In the table below, the “support failures” represents the percentage of cases where the support was eaten but the tablet was spat out, and similarly, the effectiveness corresponds to the percentage of cases where the support and the tablet were eaten. The test results are summarized in Table 1 below:

| | % for a dog | % for a cat |
|---|---|---|
| Support failures | 2.5% | 9.5% |
| Effectiveness | 90% | 80% |

As can be seen, the invention provides a highly effective support, both for dogs and for cats.

Clearly, the present invention is not limited to the embodiment described and shown by way of non-limiting illustrative example. In particular, the shape and size of the matrix, the presence and nature of the palatability enhancer, the presence of a slit, a groove, an incision, a hole, or a drain, are optional.

1-19. (canceled)

20. A palatable support intended to at least partially wrap an active substance that is initially independent of the support, and intended for a cat or a dog, wherein the support includes, as a percentage by weight based on the total weight of the support, 3% to 50% of glucose syrup, the dextrose equivalent DE of the glucose syrup being in the range 5 to 60, and at least 2% of rice starch.

21. A support according to claim 20, including at least 4% of rice starch.

22. A support according to claim 20, wherein the percentage by weight of glucose syrup, based on the total weight of the support, is more than 5% and less than 10%.

23. A support according to claim 20, wherein the dextrose equivalent is more than 10 and less than 25.

24. A support according to claim 20, including a moistening agent selected from alcohols or a mixture thereof.

25. A support according to claim 20, including as a percentage by weight based on the total weight of the support, 3% to 15% of sorbitol.

26. A support according to claim 20, including as a percentage by weight based on the total weight of the support:
in the range 3% to 15% of a mixture of propylene glycol and glycerol, the quantities of propylene glycol and glycerol being in the range 3% to 15% and in the range 5% to 20% respectively;
in the range 1% to 15% of lecithin;
less than 15% of a palatability enhancer.

27. A support according to claim 20, including as a percentage by weight based on the total weight of the support:
5% to 90% of protein of animal origin;
4% to 90% of starch;
less than 10% of fats;
0.01% to 10% of at least one preservative.

28. A support according to claim 20, comprising a mixture of preservatives including, as a percentage by weight based on the total weight of the support, 0.01% to 2% of Thermox™ to 0.5% to 5% of Pet SAVOR®.

29. A support according to claim 20, comprising proteins of animal origin selected from the group formed by poultry, pig and fish meal and mixtures thereof and/or rice starch and/or a palatability enhancer selected from Superpremium™ products.

30. A support according to claim 20, in which the rice starch is pre-hydrolyzed.

31. A support according to claim 1, in which the rice starch comprises more than 90% by weight of amylopectin.

32. A support according to the preceding claim, in which the rice starch is waxy.

33. A support according to the preceding claim, in which the rice starch is AXFGP.

34. A support, comprising, as a percentage by weight based on the total weight of the support:
in the range 41.5% to 42.5% of poultry meal;
in the range 4% to 5% of a palatability enhancer of the Superpremium™ type;
in the range 5.3% to 6.3% of hydrolyzed rice starch;
in the range 0 to 1% of salt;
in the range 0.01% to 1% of sorbate;
in the range 0.01% to 0.1% of Thermox™;
in the range 11% to 12% of glycerol;
in the range 7% to 8% of propylene glycol;
in the range 5.5% to 6.5% of glucose syrup with a dextrose equivalent DE in the range 18 to 22;
in the range 2% to 3% of oil;
in the range 0.01% to 1% of fats;
in the range 0.6% to 1.6% of Pet SAVOR®;
in the range 5.2% to 6.2% of lecithin;
in the range 10.3% to 11.3% of water;
in the range 0.01% to 1% of emulsifying agent.

35. A method of packaging an active substance within a palatable support, said active substance, intended for an animal, initially being independent of said support, in which the user at least partially wraps said active substance in said support, wherein the support comprises, as a percentage by weight based on the total weight of the support, 3% to 50% of glucose syrup, the dextrose equivalent DE of the glucose syrup being in the range 5 to 60.

36. A packaging method according to claim 35, wherein the dextrose equivalent DE of the glucose syrup is more than 15 and less than 40.

37. A packaging method according to claim 35, wherein the palatable support includes, as a percentage by weight based on the total weight of the support, 3% to 50% of glucose syrup, the dextrose equivalent DE of the glucose syrup being in the range 5 to 60, and at least 2% of rice starch.

38. A packaging method according to claim 35, wherein the palatable support as a percentage by weight based on the total weight of the support:
in the range 41.5% to 42.5% of poultry meal;
in the range 4% to 5% of a palatability enhancer of the Superpremium™ type;
in the range 5.3% to 6.3% of hydrolyzed rice starch;
in the range 0 to 1% of salt;
in the range 0.01% to 1% of sorbate;
in the range 0.01% to 0. 1% of Thermox™;
in the range 11% to 12% of glycerol;
in the range 7% to 8% of propylene glycol;
in the range 5.5% to 6.5% of glucose syrup with a dextrose equivalent DE in the range 18 to 22;
in the range 2% to 3% of oil;
in the range 0.01% to 1% of fats;
in the range 0.6% to 1.6% of Pet SAVOR®;
in the range 5.2% to 6.2% of lecithin;
in the range 10.3% to 11.3% of water;
in the range 0.01% to 1% of emulsifying agent.

39. A packaging method according to claim 35, wherein said active substance is in the form of pills, lozenges, gelules, tablets, or powder.
